(19) United States
(12) Patent Application Publication
(10) Pub. No.: US 2011/0036746 A1
(43) Pub. Date: Feb. 17, 2011

(54) Tooth rescue kit

(76) Inventor: Johnny R. Bear, New Orleans, LA (US)

Correspondence Address: KEATY LAW FIRM, LLC, 365 CANAL STREET, Suite 2410, NEW ORLEANS, LA, 70130, US

(21) Appl. No.: 12/462,851

(22) Filed: Aug. 11, 2009

(51) Int. Cl. A61F 17/00; A61F 13/00; A61F 7/10; B65D 35/00; G09B 23/28

(52) U.S. Cl. 206/572; 602/42; 607/114; 222/92; 434/263

(57) ABSTRACT

A portable compact emergency dental kit is provided for administering first aid in cases of avulsed, loose, or broken teeth. The kit contains step-by-step instructions for rendering the first aid even by persons untrained in dentistry. The kit contains a quantity of tooth-preserving fluid for temporary retaining knocked out teeth, as well as a tube of dental filling material that sets shortly after being dispensed from the tube. A quantity of gloves, sterile gauze, antiseptic mouth rinse, ice packs and antibacterial wipes is provided in the kit holder.

### BACKGROUND OF THE INVENTION

The invention relates to a kit for providing emergency dental aid. More particularly, the invention relates to a portable and compact kit, which provides the components to necessary to render assistance in an emergency situation so that persons untrained in dentistry could administer dental treatment when professional care is unavailable.

Dental problems often arise during travel or at sporting events, when the person is unable to obtain immediate treatment from a trained dentist or dental technician. A tooth may be broken, become loose, or even knocked out of its socket. When away from home, people are often reluctant to seek assistance from an unknown dentist preferring instead to wait until they can see their regular dentist. At a sporting event, such as school physical education classes or tournaments, children can suffer a tooth injury. If no immediate assistance is available, a child may loose a tooth permanently.

The dental art has developed a number of tools and medications for temporarily assisting a dental patient until such time a dental professional can attend to the tooth injury. However, these tools and medications are not regularly available in a cohesive system or kit that would allow an unprofessional to administer the first aid in situ.

The present invention contemplates elimination of drawbacks associated with prior treatment methods and provision of a portable compact kit for assistance with dental emergencies.

### BACKGROUND OF THE INVENTION

The invention relates to a kit for providing emergency dental aid. More particularly, the invention relates to a portable and compact kit which provides the components necessary to render assistance in an emergency situation so that persons untrained in dentistry could administer dental treatment when professional care is unavailable.

Dental problems often arise during travel or at sporting events, when the person is unable to obtain immediate treatment from a trained dentist or dental technician. A tooth may be broken, become loose, or even knocked out of its socket. When away from home, people are often reluctant to seek assistance from unknown dentists, preferring instead to wait until they can see their regular dentist. At a sporting event, such as school physical education classes or tournaments, children can suffer a tooth injury. If no immediate assistance is available a child may lose the tooth permanently.

The dental arts have developed a number of tools and medications for temporary assisting a dental patient until such time as a dental professional can attend to the tooth injury. However, these tools and medications are not readily available in a cohesive system or kit that would allow a non-professional to administer the first aid in situ.

The present invention contemplates elimination of drawbacks associated with prior treatment methods and provision of a portable compact kit for assistance with dental emergencies.

### SUMMARY OF THE INVENTION

It is, therefore, an object of the invention to provide a kit comprising a plurality of instruments, medicaments and materials assembled especially for the administration of dental aid in case of a dental emergency.

It is another object of the invention to provide an emergency dental kit that would allow a person untrained in dentistry to render assistance in cases of damage to natural teeth.

These and other objects of the invention are achieved through a provision of a portable compact emergency dental kit for administering first aid in cases of avulsed, loose, or broken teeth. The kit contains step-by-step instructions for rendering the first aid even by persons untrained in dentistry. The kit contains a quantity of tooth-preserving fluid for temporary retaining knocked out teeth, as well as a tube of dental filling material that sets shortly after being dispensed from the tube. A quantity of gloves, sterile gauze, antiseptic mouth rinse, ice packs, and antibacterial wipes is provided in the kit holder.

### BRIEF DESCRIPTION OF THE DRAWINGS

Reference will no be made to the drawings, wherein like parts are designated by like numerals, and wherein

FIG. 1 is an elevation view of a dental emergency kit in accordance with a preferred embodiment of the invention.

FIG. 2 illustrates an instruction card for assisting with avulsed tooth.

FIG. 3 illustrates an instruction card for assisting with a loose tooth.

FIG. 4 illustrates an instruction card for assisting with a chipped or broken tooth.

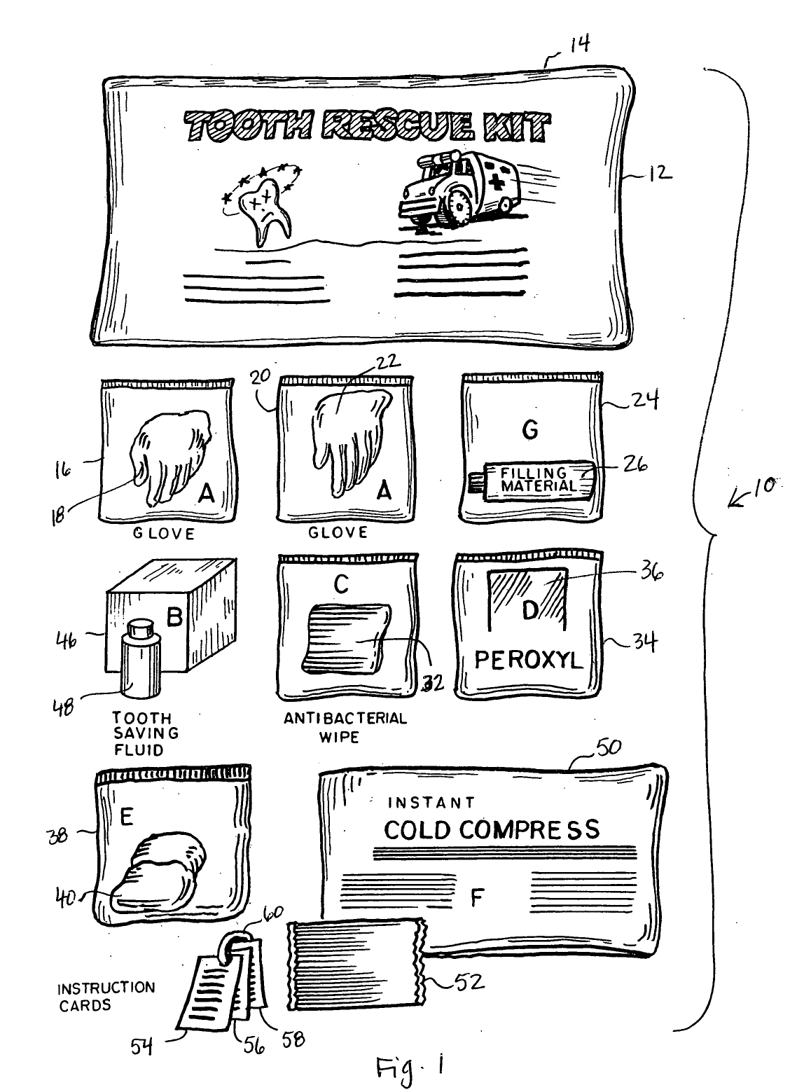

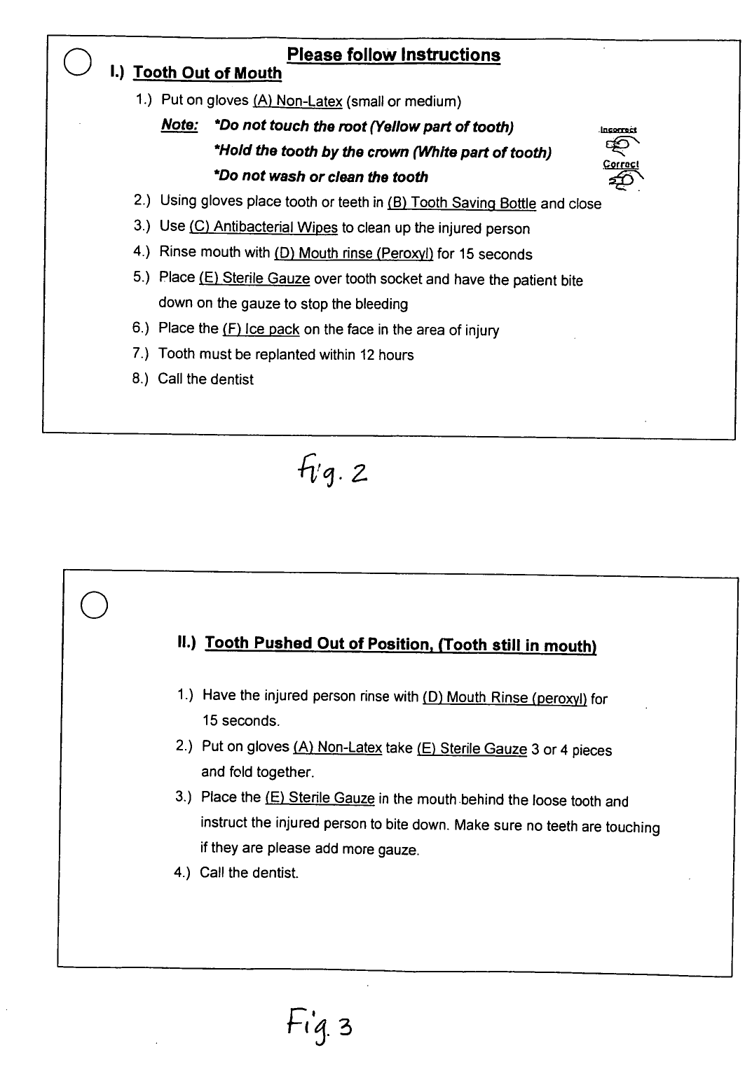

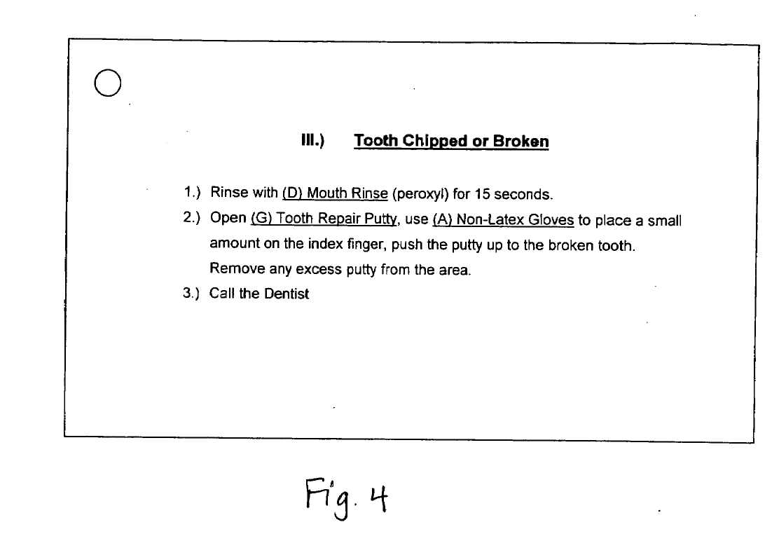

### DETAIL DESCRIPTION OF THE INVENTION

Turning now to the drawings in more detail, numeral 10 designates the emergency dental kit in accordance with the present invention. The kit 10 comprises a plurality of articles, the articles comprising chemical compounds and medical instruments, and a holder for the articles configured to hold all articles in a compact portable manner.

To this end, the present invention provides for a holder 12 that can be conveniently closed and opened by sliding a zipper 14 located in an upper part of the holder 12. Alternatively, the zipper 12 can be replaced with a sliding clasp fastener, or other means for closing the holder 12. The holder can be made of flexible, deformable, resilient material in a size and shape to accommodate the emergency dental articles.

The articles included in the kit 10 comprise a plurality of small pouches and boxes containing various dental assistance supplies. The pouches can be formed from a transparent sheet of plastic and have a sliding lock tops. The pouches are reclosable; they can be conventional plastic bags having mating plastic seals or so-called “zip locks.”

A first pouch 16 is configured to retain a pre-determined quantity of disposable gloves 18, which may be latex or non-latex gloves. The gloves 18 are of a pre-determined size, for instance small size, to accommodate smaller hands. A second pouch 20 is configured to retain a pre-determined quantity of gloves 22, which can be of a different size than gloves 18, for instance medium size. The pouches 16 and 20 bear indicia, or label with the letter “A,” the purpose of which will be explained hereinafter.

A third pouch 24 is configured to hold a tube 26 of a temporary filling material, or cement. The encapsulated filling material may be a mixture of zinc oxide powder and eugenol. The mixture is pasty or pliable inside the tube 26 but sets within a few minutes into a relatively hard compound suitable for use as a dental cement and temporary filling material after being squeezed out of the tube 26. The self-hardening dental filling material may contain other components, depending on the manufacturer's formula. For identification purposes, the third pouch 24 is labeled with the letter “G.”

The fourth pouch 30 is configured to retain a pre-determined quantity of encapsulated antibacterial wipes 32, each of which is individually packaged. For identification purposes, the third pouch 30 bears the letter “C.”

The fifth pouch 34 contains a small bottle or vial 36 of an antiseptic mouth rinse, such as for instance Peroxyl. The mouth rinse is designed to reduce bacteria in minor oral wounds to help prevent infection. The pouch 30 with the mouth rinse is identified with a label “D.”

The sixth pouch 38 contains a pre-determined quantity of sterile gauze pieces 40, which may be individually wrapped to protect the sterile environment. The pouch 38 is labeled with the letter “E.”

A first box 46 is configured to house a small bottle of a pH-balanced cell-culture fluid which is biocompatible with the tooth's PDL (Periodontal Ligament) cells. Such compound is manufactured by Biochrom AG of Germany and sold under the name “EMT Tooth Saver.” The bottle 48 has a relatively large open top to allow an avulsed tooth to be placed in the bottle. A screw-on cap closes the open top of the bottle 48. Once inside, the tooth can be safely stored up to 24 hours until reimplantation. The first box 46 is labeled with the letter “B.”

The second box 50 is configured to retain one or more individually-sealed instant cold compresses 52. The instant cold compress (ice pack) 52 is adapted to be placed at the site of the injury to temporarily relieve pain and swelling of the tissue. The instant cold compress 52 is disposable with no pre-chilling required. The instant compress 52 may contain ammonium nitrate and water that are stored in separate compartments (not shown) while inside the outer cover of the encapsulated ice pack 52. To activate the cold pack, a user needs to press or crush the individually-sealed packages 52. The box 50 is labeled with the letter “F.” The boxes 46 and 50 may be made of paperboard or cardboard material.

The kit 10 further comprises a plurality of instruction cards 54, 56, and 58, which are secured together by a snap ring 60. The cards 54, 56, and 58 may be formed of paperboard, cardboard or plastic with the instructions for a non-professional caregiver imprinted therein.

The instruction card 54 provides instructions to the user of the steps that need to be taken in case when the tooth has been knocked out of the mouth. The instruction card 54 describes the procedure requiring the caregiver to put on gloves taken from one of the pouches A. The card 54 also directs the caregiver to avoid touching the root of the tooth, while placing the tooth into the bottle in the box B. Further instructions on the card 54 advise the caregiver to clean the injured person using the antibacterial wipes stored in the pouch C.

The injured person is then directed to rinse his/her mouth with the mouth rinse for 15 seconds, after which to place a sterile gauze from the container E over the tooth socket, while the patient bits down on the gauze to stop bleeding. The ice pack F can then be placed on the injured person's face in the area of injury. The card 54 also instructs to call the doctor within 12 hours for tooth reimplantation.

The instruction card 56 provides instructions to the caregiver in cases when the tooth is still in the mouth of the injured person but has been pushed out of its normal position that is became loose. The card 56 instructs to have the injured person rinse the mouth with the mouth rinse for 15 seconds; then put on gloves from the pouches labeled A and remove 3 or 4 sterile gauze pieces from the pouch labeled E. The user is then instructed to fold the gauze pieces together and place them in the mouth of the injured person behind the loose tooth. The injured person is then instructed to bite down on the gauze pieces to stop bleeding, while making sure that the teeth are not touching. The injured person is then instructed to call the doctor.

The card 58 contains instructions for occasions when the tooth appears to be chipped or broken. The injured person is instructed to rinse the mouth with the mouth rinse from the container D for 15 seconds. The user is then instructed to squeeze a small quantity of the tooth filling material from the tube in pouch G and place the putty on the gloved index finger. The user is also instructed to push the putty to the broken tooth and then remove any excess putty from the area. The user is also instructed to call the doctor.

To facilitate use of the kit 10 by a person not having training in dental procedures and equipment, all components of the kit 10 are preferably packaged in reclosable bags with large labels bearing indicia corresponding to the letter codes in the instructions cards. The instruction cards 54, 56, and 58, along with the other articles of the kit are removably insertable into the holder 12.

Each of the kit components is designed to provide the means necessary to treat a specific dental emergency. The kit contains clear, easily understood instructions to enable identification of the problem and to treat the patient simply and effectively, while relieving the discomfort of the injured person, until professional dental treatment can be obtained.

Many changes and modifications can be made in the present invention without departing from the spirit thereof. I, therefore, pray that my rights to the present invention be limited only by the scope of the appended claims.

I claim:

1. A kit for providing emergency dental aid comprising:
a holder configured to retain a plurality of first aid articles, said articles comprising gloves, an encapsulated quantity of dental filling material, a reclosable container with a pre-determined quantity of pH-balanced cell-culture fluid suitable for temporary preserving avulsed teeth, a plurality of sterile gauze pieces, a quantity of encapsulated antibacterial wipes, a pre-determined quantity of antiseptic mouth rinse, a quantity of encapsulated cold compresses, and instruction cards for providing emergency dental aid.

2. The kit of claim 1, wherein each of the articles is enclosed in a container with labeled indicia that corresponds to indicia on the instruction cards.

3. The kit of claim 1, wherein the gloves, the encapsulated dental filling, the antibacterial wipes, the quantity of mouth rinse and the sterile gauze are positioned in resealable plastic bags.

4. The kit of claim 1, wherein the dental filling material is encapsulated in a tube.

5. The kit of claim 1, wherein the reclosable container for pH-balanced cell-culture fluid is a bottle.

6. The kit of claim 1, wherein the instruction cards carry indicia thereon for providing instructions in such emergencies as avulsed teeth, loose teeth, and broken teeth.

7. The kit of claim 7, wherein the indicia comprises step-by-step instructions for administering first aid by a person unskilled in dentistry.

8. The kit of claim 1, wherein the encapsulated cold compresses comprise an instant cold pack.

9. A kit for providing emergency dental aid comprising:
a holder configured to retain a plurality of first aid articles, said articles comprising different size gloves, a tube with a quantity of self-hardening dental filling material, a reclosable container with a pre-determined quantity of pH-balanced cell-culture fluid suitable for temporary preserving avulsed teeth, a plurality of sterile gauze pieces, a quantity of encapsulated antibacterial wipes, a pre-determined quantity of antiseptic mouth rinse, a quantity of encapsulated cold compresses, and instruction cards for providing emergency dental aid.

10. The kit of claim 9, wherein the gloves, the tube with a pre-determined quantity of dental filling, the antibacterial wipes, the quantity of mouth rinse and the sterile gauze are positioned in resealable transparent plastic bags.

11. The kit of claim 9, wherein each of the articles is enclosed in a container with labeled indicia that corresponds to indicia on the instruction cards.

12. The kit of claim 9, wherein the reclosable container for pH-balanced cell-culture fluid is a bottle.

13. The kit of claim 9, wherein the instruction cards carry indicia thereon for providing instructions in such emergencies as avulsed teeth, loose teeth, and broken teeth.

14. The kit of claim 13, wherein the indicia comprises step-by-step instructions for administering first aid by a person unskilled in dentistry.

15. The kit of claim 9, wherein the encapsulated cold compresses comprise an instant cold pack.

16. The kit of claim 9, wherein the kit is portable.
